(19) United States
(12) Patent Application Publication
(10) Pub. No.: US 2010/0093060 A1
(43) Pub. Date: Apr. 15, 2010

(54) GROWTH OF MICROORGANISMS IN CELLULOSIC MEDIA

(76) Inventors: Kevin A. Jarrell, Lincoln, MA (US); Gabriel Reznik, Brookline, MA (US); Michelle A. Pynn, Sharon, MA (US); Joy D. Sitnik, Dracut, MA (US)

Correspondence Address: CHOATE, HALL & STEWART LLP, TWO INTERNATIONAL PLACE, BOSTON, MA, 02110, US

(21) Appl. No.: 12/565,408

(22) Filed: Sep. 23, 2009

Related U.S. Application Data
(60) Provisional application No. 61/099,227, filed on Sep. 23, 2008.
(60) Provisional application No. 61/117,877, filed on Nov. 25, 2008.

(51) Int. Cl. C12N 1/20; C12N 1/22

(52) U.S. Cl. 435/252.5; 435/253.6

(57) ABSTRACT

The present invention provides novel methods of growing of microorganisms in cell culture media comprising cellulosic material as a carbon source. The present invention further provides novel cell culture media cellulosic material as a carbon source. In certain embodiments, inventive cell culture media substantially lack a carbon source other than cellulosic material (e.g., the media substantially lack glucose and glycerol). In certain embodiments, inventive cell culture media comprise cellulosic material as the sole carbon source.

### CROSS REFERENCE TO RELATED APPLICATIONS

The present application is copending with, shares at least one common inventor with and claims priority to U.S. provisional patent application Ser. No. 61/099,227, filed Sep. 23, 2008, and to U.S. Provisional Application No. 61/117,877, filed Nov. 25, 2008. The entire contents of the prior applications are herein incorporated by reference.

### BACKGROUND

Microorganisms are typically grown in cell culture media that contain a carbon source. Carbon sources are often simple sugars such as glucose or galactose, which are broken down and converted to energy, cellular components, and/or metabolic products. The choice of which carbon source to use in the culturing of microorganisms is determined by a variety of factors such as the ability of the microorganism to utilize a particular carbon source, the ability of the microorganism to convert a particular carbon source into a product of interest, the type and amount of byproducts produced as a result of metabolizing the carbon source, the availability of a carbon source, the present and/or future cost a particular carbon source, etc.

In some cases, microorganisms are grown in cell culture media that contain glucose or refined glycerol as an energy source. Glucose is commercially produced by enzymatic hydrolysis of starches derived from crops such as maize, rice, wheat, potato, cassaya, arrowroot, and sago. Refined glycerol is typically generated from crude glycerol through an intensive process that removes contaminants and impurities that are generally thought to be detrimental to the growth of microorganisms. Less expensive, renewable alternative carbon sources are needed for economical and sustainable commercial-scale production of compounds produced by microorganisms.

### SUMMARY OF THE INVENTION

The present invention provides improved compositions and methods for growing microorganisms (e.g., bacteria or fungi) in cell culture media using cellulosic carbon sources (e.g., inexpensive cellulosic materials such wood waste, paper waste, or agricultural plant waste, e.g., saw dust or soybean hulls, or cellobiose, xylose, or xylan). In certain embodiments, methods are provided wherein a microorganism is grown in a cell culture comprising a cellulosic carbon source. In certain embodiments, methods are provided wherein a microorganism is grown in a cell culture comprising a cellulosic carbon source, which cell culture further substantially lacks added glucose and/or glycerol (e.g., refined glycerol). In certain embodiments, methods are provided wherein a microorganism is grown in a cell culture comprising cellulosic material as the sole carbon source.

The present invention provides culture media suitable for growth of microorganisms. In certain embodiments, a cell culture medium of the present invention comprises a cellulosic carbon source. In certain embodiments, a cell culture medium of the present invention comprises a cellulosic carbon source, which cell culture medium further substantially lacks added glucose and/or glycerol. In certain embodiments, a cell culture medium of the present invention comprises cellulosic material as the sole carbon source.

In certain embodiments, a cell culture medium comprises a cellulosic carbon source (e.g., an unprocessed cellulosic material, or a processed and/or purified cellulosic material) at a weight to volume ratio of 1, 2, 3, 4, 5, 6, 7, 8, 9, 10, 11, 12, 13, 14, or 15%. In some embodiments, a cell culture medium comprises a cellulosic carbon source at a weight to volume ratio of 2-10%. In certain embodiments, a cell culture comprises a cellulosic carbon source at a weight to volume ratio of 2-8%. In certain embodiments, a cell culture medium comprises a cellulosic carbon source at a weight to volume ratio of 1-15% (e.g., 2-10%, or 2-8%), and includes less than 0.1% of a non-cellulosic carbon source, such as glucose.

In some embodiments, a cell culture medium comprising a cellulosic carbon source lacks an exogenous carbohydrase. In some embodiments, a cell culture medium comprises an exogenous carbohydrase, e.g., one or more of cellulase, cellobiase, hemicellulase, and pectinase. In some embodiments, a culture medium is a liquid medium. In some embodiments, a culture medium is a solid medium.

Any of a wide variety of microorganisms can be grown in inventive cell culture media that comprise cellulosic material as a carbon source. For example, any of a variety of bacteria may be grown according to the present invention. As non-limiting examples, bacteria of the genera Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix and Actinosynnema may be grown in accordance with compositions and/or methods of the present invention. In certain embodiments, a bacterium of the genus Bacillus grown is grown in accordance with compositions and/or methods of the present invention. In certain embodiments, a bacterium of the species Bacillus subtilis is grown in accordance with compositions and/or methods of the present invention.

Additionally or alternatively, any of a variety of fungi may be grown according to the present invention. In certain embodiments, a fungus grown in accordance with compositions and/or methods of the present invention is a yeast. As non-limiting examples, yeast of the genera Saccharomyces, Pichia, Aspergillus, Trichoderma, Kluyveromyces, Candida, Hansenula, Schizpsaccaromyces, Yarrowia, Chrysoporium, Rhizopus, Aspergillus and Neurospora may be grown in accordance with compositions and/or methods of the present invention. In certain embodiments, a yeast of the genus Saccharomyces grown is grown in accordance with compositions and/or methods of the present invention. In certain embodiments, a yeast of the species Saccharomyces cerevisiae is grown in accordance with compositions and/or methods of the present invention.

Microorganisms grown in a cell culture medium described herein can be used to produce any of a variety of products. In certain embodiments, a microorganism grown in an inventive cell culture medium and/or according to inventive methods produces a polypeptide, non-ribosomal peptide, acyl amino acid, and/or lipopeptide of interest (e.g., an acyl amino acid or lipopeptide which is a surfactant). As one non-limiting example, a microorganism grown in an inventive cell culture medium and/or according to inventive methods may produce surfactin. As another example, a microorganism grown in an inventive cell culture medium and/or according to inventive methods may produce acyl glutamate. Those of ordinary skill in the art will be aware of other polypeptides, non-ribosomal peptides, and/or lipopeptides of interest, as well as microorganisms that produce them. Such art-recognized polypeptides, non-ribosomal peptides, acyl amino acids, and/or lipopeptides of interest can be grown in inventive cell culture media and/or according to methods of the present invention. In certain embodiments, such a microorganism produces the polypeptide, non-ribosomal peptide, acyl amino acid, and/or lipopeptide of interest to a level that is at least that of a microorganism grown in traditional cell culture media and/or according to traditional methods. In certain embodiments, the yield (defined as percent of carbon source converted into a product of interest) of a polypeptide, non-ribosomal peptide, acyl amino acid, and/or lipopeptide of interest produced by a microorganism grown in inventive media containing cellulosic material is at least about 10%, 15%, 20%, 25%, 30%, 35%, 40%, 45%, 50%, 55%, 56%, 57%, 58%, 59% 60% or more. In some embodiments, a microorganism grown in a medium described herein expresses a recombinant polypeptide which produces a product of interest. For example, in some embodiments, a microorganism is engineered to express a polypeptide that produces an acyl amino acid, e.g., acyl glutamate.

In certain embodiments, a microorganism grown in an inventive cell culture medium and/or according to inventive methods that produces a polypeptide, non-ribosomal peptide, acyl amino acid, and/or lipopeptide of interest is a bacterium. As non-limiting examples, bacteria of the genera Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix and Actinosynnema may be grown in accordance with compositions and/or methods of the present invention to produce a polypeptide, non-ribosomal peptide, acyl amino acid, and/or lipopeptide of interest. In certain embodiments, such a bacterium is of the genus Bacillus. In certain embodiments, such a bacterium is of the species Bacillus subtilis.

In certain embodiments, an inventive cell culture medium comprises a nitrogen source. Nitrogen sources that can be used in accordance with the present invention include, but are not limited to, tryptone, total soy extract, yeast extract, casamino acids and/or distiller grains.

In another aspect, the present invention provides methods for growing microorganisms (e.g., fungi or bacteria, e.g., Bacillus cells, such as Bacillus subtilis cells) in a cell culture, the method comprising growing the cells in a cell culture medium comprising a carbon source which comprises cellulosic material. In some embodiments, the cellulosic material comprises wood waste, paper waste, or agricultural plant waste such as sawdust or soybean hulls. In some embodiments, the cellulosic material comprises cellobiose, xylose, or xylan. In certain embodiments, the medium includes less than 0.1% glucose. In some embodiments, the medium lacks a carbon source other than the cellulosic material.

The microorganisms can include microorganisms that produce a product. In some embodiments, microorganisms produce a lipopeptide or an acyl amino acid. In some embodiments, cells (e.g., Bacillus cells) comprise a recombinant polypeptide which produces a lipopeptide or acyl amino acid. In some embodiments, a recombinant polypeptide produces acyl glutamate.

In some embodiments, cells produce a lipopeptide which comprises surfactin. In some embodiments, the yield of surfactin produced from a cell culture is at least about 40 mg/L, 50 mg/L, 75 mg/L, 100 mg/L, 0.2 g/L, 0.3 g/L, 0.5 g/L, 0.7 g/L, 0.9 g/L or 1 g/L.

In some embodiments, medium used in a cell culture has less than 0.1% glucose, and the cell culture produces a lipopeptide or acyl amino acid at a level at least comparable to a level of the lipopeptide or acyl amino acid produced in a culture in a medium having added glucose and which is otherwise identical to the medium. In some embodiments, a medium comprises a cellulosic material at a weight to volume ratio of 1-15% (e.g., 1-10%, or 2-8%). In some embodiments, a medium is a liquid medium. In some embodiments, a medium is a solid medium.

In some embodiments, a medium comprises one or more of (NH₄)₂SO₄, K₂HPO₄, KH₂PO₄, Na₃-citrate dihydrate, magnesium sulfate heptahydrate, CaCl₂ dihydrate, FeSO₄ heptahydrate, and disodium EDTA dihydrate (e.g., a medium comprises: (NH₄)₂SO₄ at a concentration of about 2 g/L; K₂HPO₄ at a concentration of about 14 g/L; KH₂PO₄ at a concentration of about 6 g/L; Na₃-citrate dihydrate at a concentration of about 1 g/L; magnesium sulfate heptahydrate at a concentration of about 0.2 g/L; CaCl₂ dihydrate at a concentration of about 14.7 mg/L; FeSO₄ heptahydrate at a concentration of about 1.1 mg/L; and disodium EDTA dihydrate at a concentration of about 1.5 mg/L). In some embodiments, a medium further comprises MnSO₄ (e.g., at a concentration of about 10 μM).

In some embodiments, a medium comprises a nitrogen source selected from the group consisting of: total soy extract, tryptone, yeast extract, casamino acids, distiller grains, and combinations thereof.

The present invention provides methods of producing a lipopeptide or an acyl amino acid. Methods include, for example, providing a cell culture by growing cell (e.g., Bacillus cells) that produce a lipopeptide or an acyl amino acid in a cell culture medium, wherein the medium comprises a carbon source which comprises cellulosic material; thereby producing a lipopeptide or acyl amino acid. Methods can further include isolating a portion of the cell culture which comprises the lipopeptide or acyl amino acid. Methods can further include purifying the lipopeptide or acyl amino acids.

The present invention provides methods of producing a lipopeptide or an acyl amino acid. Methods include, for example, providing a first cell culture by growing cells (e.g., Bacillus cells) that produce a lipopeptide or an acyl amino acid in a first cell culture medium, wherein the first medium comprises glycerol or glucose as a carbon source; providing a second cell culture by inoculating a second cell culture medium with a portion of the first cell culture, wherein the second medium comprises cellulosic material as a carbon source; thereby producing a lipopeptide or acyl amino acid. In some embodiments, the first cell culture is grown for about 24 hours prior to inoculating the second culture.

The present invention also provides compositions including microorganisms and a cell culture medium described herein, as well as compositions that include a product produced by the microorganisms. For example, the invention provides a composition comprising Bacillus cells and a cell culture medium, wherein the cell culture medium comprises a carbon source which comprises cellulosic material. In some embodiments, the cellulosic material comprises one or more of soybean hulls, cellobiose, xylose, or xylan. In some embodiments, the Bacillus cells produce a lipopeptide or acyl amino acid. Also provided are compositions comprising lipopeptides and/or acyl amino acids produced by the cells.

The details of one or more embodiments of the invention are set forth in the accompanying drawings and the description below. Other features, objects, and advantages of the invention will be apparent from the description and drawings, and from the claims. All cited patents, patent applications, and references (including references to public sequence database entries) are incorporated by reference in their entireties for all purposes.

### BRIEF DESCRIPTION OF THE DRAWING

FIG. 1 is a graph depicting surfactin production (grams/liter) for Bacillus production cultures including glucose (4% w/v), xylose (4%, 8%, 16%, or 42% w/v), cellobiose (1.9%, 3.8%, or 10.2% w/v) or xylan (1%, 4%, or 10% w/v).

FIG. 2 is a schematic depiction of the structure of a chimeric enzyme with the first module of SRFA-A (the L-Glu module) linked to the thioesterase domain (TE). P_{srfa}, surfactin promoter; C, condensation domain; A, adenylation domain; T, thiolation domain; TE, thioesterase.

FIG. 3 shows the structure of β-hydroxy myristoyl glutamate, FA-Glu, the acyl amino acid synthesized by the FA-Glu enzyme depicted in FIG. 2.

FIG. 4 shows results of mass spectrometry (MS) analysis of lipopeptides isolated from culture media of an FA-Glu strain. The mass spectrum identifies both monomers and homodimers of an FA-Glu acyl amino acid.

FIG. 5 is a purification flowchart for FA-Glu. FA-Glu was produced in a fermentor with an 9-liter working volume. LC-MS was used to monitor each step of the purification.

FIG. 6 is a graph depicting results used to determine the Critical Micelle Concentration for FA-Glu () and myristoyl glutamate (⋄).

FIG. 7 is a graph showing production of FA-Glu by fermentation of cellulosic material. Results from simultaneous saccharification and fermentation (SSF) of soybean hulls are shown. C, Cellulase; B, Cellobiase; H, Hemicellulase; P, Pectinase.

FIG. 8 is a graph showing production of FA-Glu by fermentation of cellulosic material. Results from fermentation of purified carbohydrates of lignocellulosic origin (cellobiose, xylose) and glucose are shown.

### DETAILED DESCRIPTION OF CERTAIN EMBODIMENTS

### Definitions

“Acyl amino acid”: The term “acyl amino acid” as used herein refers to an amino acid that is covalently linked to a fatty acid. In certain embodiments, acyl amino acids are produced in microorganisms expressing engineered polypeptides, e.g., engineered polypeptides comprising a peptide synthetase domain covalently linked to a fatty acid linkage domain and a thioesterase domain or reductase domain. In certain embodiments, acyl amino acids are produced in microorganisms expressing engineered polypeptides comprising a peptide synthetase domain covalently linked to a beta-hydroxy fatty acid linkage domain and a thioesterase domain. In certain embodiments, acyl amino acids are produced in microorganisms expressing engineered polypeptides comprising a peptide synthetase domain covalently linked to a beta-hydroxy fatty acid linkage domain and a reductase domain. In certain embodiments, an acyl amino acid produced by a method described herein comprises a surfactant such as, without limitation, an acylated glutamate, e.g., cocoyl glutamate. In certain embodiments, acyl amino acids produced by compositions and methods of the present invention comprise a beta-hydroxy fatty acid. A beta-hydroxy fatty acid may contain 3, 4, 5, 6, 7, 8, 9, 10, 11, 12, 3, 14, 15, 15, 16, 17, 18, 19, 20 or more carbon atoms. In some embodiments, a beta-hydroxy fatty acid is beta-hydroxy myristic acid, which contains 13 to 15 carbons in the fatty acid chain.

“Carbon source”: The term “carbon source” as used herein refers to a component of a cell culture medium that comprises carbon and that is utilized by a cell (e.g., a microbial cell) in culture medium for producing energy, cellular components, and/or metabolic products. Examples of carbon sources used in cell culture media include sugars, carbohydrates, organic acids, and alcohols (e.g., glucose, fructose, mannitol, starch, starch hydrolysate, cellulosic materials, cellulose hydrolysate, molasses, soy molasses, acetic acid, propionic acid, lactic acid, formic acid, malic acid, citric acid, fumaric acid, glycerol, inositol, mannitol and sorbitol).

“Cellulosic material”: As used herein, the term “cellulosic material” refers to any type of composition that includes cellulosic carbohydrates from plant biomass, such as cellulose, hemicellulose (e.g., xylan, xyloglucan, arabinoxylan, arabinogalactan, glucuronoxylan, glucomannan and galactomannan), xylose, cellobiose, pectin, fucose, and apiose. In some embodiments, a cellulosic material includes at least 10%, 15%, 20%, 25%, 30%, 35%, 40%, 45%, or 50% cellulose, hemicellulose, and/or a decomposition product thereof, such as xylan, cellobiose, or xylose. Cellulosic material can include grass, paper, paper waste, paper pulp, wheat straw, soybean hulls, leaves, cotton seed hairs, corn cobs, hardwood stems, softwood stems, sawdust or other wood waste, nut shells, combinations thereof, and processed fractions thereof. Exemplary plant sources for cellulosic materials include soybeans, sugar cane, corn, wheat, rice, grasses (e.g., Miscanthus, Switchgrass, Bermuda grass, and/or Elephant grass), woody plants or trees. In some embodiments, a cellulosic material in a composition or method described herein is sterilized prior to use (e.g., by autoclaving).

“Crude glycerol”: The term “crude glycerol” as used herein refers to glycerol that has not been subjected to art-recognized processes that remove contaminants and/or impurities to generate “refined glycerol” (see definition of “refined glycerol”, infra). Crude glycerol is produced by a variety of natural and synthetic processes. For example, crude glycerol is produced during the process of biodiesel production. Additionally, crude glycerol is produced during the process of saponification (e.g., making soap or candles from oils or fats). Crude glycerol may be subjected to one or more processes to render it suitable and/or more advantageous for use in growing microorganisms without converting it to “refined glycerol” as the term is used herein. For example, crude glycerol may be autoclaved to sterilize it. Additionally or alternatively, crude glycerol may be subjected to a filtration step to remove solids and other large masses. Such filtration can be performed on crude glycerol itself of on a culture medium that comprises crude glycerol. Crude glycerol subjected to such processes is not “refined glycerol” as the term is used herein.

“Culture medium”: The term “culture medium” as used herein refers to any type of medium suitable for growth of a cell (e.g., a cell of a microorganism, e.g., a bacterial cell and/or a fungal cell). In some embodiments, a culture medium comprises medium in liquid form. In some embodiments, a culture medium comprises medium in solid form (e.g., solid agar).

“Lipopeptide”: The term “lipopeptide” as used herein refers to any of a variety of molecules that contain a peptide backbone covalently linked to one or more fatty acid chains. Often, lipopeptides are produced naturally by certain microorganisms. Lipopeptides can also be produced in microorganisms that are engineered to express the lipopeptides. A lipopeptide is typically produced by one or more nonribosomal peptide synthetases that build an amino acid chain without reliance on the canonical translation machinery. For example, surfactin is cyclic lipopeptide that is naturally produced by certain bacteria, including the Gram-positive endospore-forming bacteria Bacillus subtilis. Surfactin consists of a seven amino acid peptide loop, and a hydrophobic fatty acid chain (beta-hydroxy myristic acid) thirteen to fifteen carbons long. The fatty acid chain allows permits surfactin to penetrate cellular membranes. The peptide loop is composed of the amino acids glutamic acid, leucine, D-leucine, valine, aspartic acid, D-leucine and leucine. Glutamic acid and aspartic acid residues at positions 1 and 5 respectively, constitute a minor polar domain. On the opposite side, valine residue at position 4 extends down facing the fatty acid chain, making up a major hydrophobic domain. Surfactin is synthesized by the linear nonribosomal peptide synthetase, surfactin synthetase is synthesized by the three surfactin synthetase subunits SrfA-A, SrfA-B, and SrfA-C. Each of the enzymes SrfA-A and SrfA-B consist of three amino acid activating modules, while the monomodular subunit SrfA-C adds the last amino acid residue to the heptapeptide. Additionally the SrfA-C subunit includes the thioesterase domain (“TE domain”), which catalyzes the release of the product via a nucleophilic attack of the beta-hydroxy of the fatty acid on the carbonyl of the C-terminal Leu of the peptide, cyclizing the molecule via formation of an ester. Other lipopeptides and their amino acid and fatty acid compositions are known in the art, and can be produced in accordance with compositions and/or methods of the present invention. In certain embodiments, lipopeptides are produced by a method described herein in microorganisms engineered to express one or more polypeptides that participate in lipopeptide synthesis. In certain embodiments, lipopeptides produced by compositions and methods of the present invention comprise a beta-hydroxy fatty acid. A beta-hydroxy fatty acid may contain 3, 4, 5, 6, 7, 8, 9, 10, 11, 12, 3, 14, 15, 15, 16, 17, 18, 19, 20 or more carbon atoms. In some embodiments, a beta-hydroxy fatty acid is beta-hydroxy myristic acid, which contains 13 to 15 carbons in the fatty acid chain.

“Nitrogen source”: The term “nitrogen source” as used herein refers to a component of a cell culture medium that comprises nitrogen and is utilized by a cell (e.g., a microbial cell) in culture medium for growth. Examples of nitrogen sources include soy extract, tryptone, yeast extract, casamino acids, distiller grains, ammonia and ammonium salts (e.g., ammonium chloride, ammonium nitrate, ammonium phosphate, ammonium sulfate, ammonium acetate), urea, nitrate, nitrate salts, amino acids, fish meal, peptone, corn steep liquor, and the like.

“Non-ribosomal peptide”: The term “non-ribosomal peptide” as used herein refers to a peptide chain produced by one or more nonribosomal peptide synthetases. Thus, as opposed to “polypeptides” (see definition, infra), non-ribosomal peptides are not produced by a cell's ribosomal translation machinery. Polypeptides produced by such nonribosomal peptide synthetases may be linear, cyclic or branched. Numerous examples of non-ribosomal peptides that are produced by one or more nonribosomal peptide synthetases are known in the art. One non-limiting example of non-ribosomal peptides that can be produced in accordance with the present invention is surfactin. Those of ordinary skill in the art will be aware of other non-ribosomal peptides that can be produced using compositions and methods of the present invention. In certain embodiments, a non-ribosomal peptide contains one or more covalently-linked fatty acid chains and is referred to herein as a lipopeptide (see definition of “lipopeptide”, supra).

“Polypeptide”: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. For example, a polypeptide may be glycosylated. A polypeptide can comprise two or more polypeptides that function as a single active unit.

“Refined glycerol”: The term “refined glycerol” as used herein refers to glycerol is produced by subjecting crude glycerol (see definition of “crude glycerol”, supra) to art-recognized processes that remove contaminants and/or impurities. Refined glycerol is typically sold as a product that is at least 99.5% pure, although it will be recognized by those of ordinary skill in the art that the purity of refined glycerol may be lower that 99.5%. Processes to produce refined glycerol depend substantially on the type of impurities present in crude glycerol. For example, when crude glycerol is generated by hydrolysis, the starting crude glycerol is likely to be nearly 85% water, and multi-stage evaporators constructed of stainless steel are typically employed for concentration. Crude glycerol produced by other processes often has high salt content, and thin-film distillation is frequently employed. A summary containing some common purification processes is provided in Ullman's Encyclopedia of Chemical Technology, Vol. A-12, pages 480-483. Crude glycerol can also be produced as a byproduct of both biodiesel production and saponification. In both biodiesel production and saponification, the crude glycerol byproduct is subjected to one or more processes that remove contaminants and/or impurities to generate “refined glycerol”. As is known to those of ordinary skill in the art, such processes are laborious and time-consuming. “Crude glycerol” as the term is used herein refers to unprocessed or minimally processed glycerol that contains these and other contaminants and/or impurities. Removal of these contaminants and/or impurities results in what is defined herein as “refined glycerol”.

“Substantially lacks”: The term “substantially lacks” as used herein refers to the qualitative condition of exhibiting total or near-total absence of a particular component. One of ordinary skill in the biological arts will understand that biological and chemical compositions are rarely, if ever, 100% pure. Conversely, one of ordinary skill in the biological arts will understand that biological and chemical compositions are rarely, if ever, 100% free if a particular component. The term “substantially lacks” is therefore used herein to capture the concept that a biological and chemical composition may comprise a small, inconsequential amount of one or more impurities. To give but one particular example, when it is said that a cell culture medium “substantially lacks” a given component, it is meant to indicate that although a minute amount of that component may be present (for example, as a result of being an impurity and/or a breakdown product of one or more components of the cell culture medium, or as a result of being a minor component of a pre-seed culture which is inoculated into a seed or production culture), that component is nevertheless an inconsequential part of the cell culture medium and does not alter the basic properties of that cell culture medium. In certain embodiments, the term “substantially lacks”, as applied to a given component of a cell culture medium, refers to condition wherein the cell culture medium comprises less that 5%, 4%, 3%, 2%, 1%, 0.9%, 0.8%, 0.7%, 0.6%, 0.5%, 0.4%, 0.3%, 0.2%, 0.1% or less of that component. In certain embodiments, the term “substantially lacks”, as applied to a given component of a cell culture medium, refers to condition wherein the cell culture medium lacks any detectable amount of that component.

### Carbon Sources

All living organisms require a carbon or energy source for growth, production of biologically useful molecules and metabolic activity generally. Microorganisms are known to utilize a wide variety of carbon sources, many of which are simple monosaccharide and disaccharide sugars such as, for example, glucose, dextrin, lactose, sucrose, maltose, fructose, and/or mannose. Additionally or alternatively, microorganisms are known to utilize a wide variety of non-sugar carbon sources such as, for example, starch and amino acids such as glutamate.

Although each of the carbon sources listed above is used to grow microorganisms, those of ordinary skill in the art do not employ each of these carbon sources to the same extent. For example, glucose is a common carbon source for use in growing microorganisms. In addition to cost and availability, the choice of which carbon source to use in the culturing of microorganisms is determined by a variety of other factors including considerations such as the ability of the microorganism to utilize a particular carbon source, the ability of the microorganism to convert a particular carbon source into a product of interest, the type and amount of byproducts produced as a result of metabolizing the carbon source, etc. Clearly, having more options as to which carbon source to use will provide the practitioner more flexibility in choosing an appropriate and/or advantageous carbon source, depending on his or her practical, experimental, commercial and/or other needs.

Cellulosic feedstocks are an abundant, low cost, renewable potential carbon source for fermentation. The present invention encompasses the recognition that cellulosic material, e.g., low cost, renewable, abundant cellulosic material derived from sources such as wood waste (e.g., sawdust) and soybean waste (e.g., soybean hulls), can be used as a carbon source, and even as a sole carbon source, for the growth of microorganisms, e.g., for the production of products such as polypeptides, non-ribosomal peptides, acyl amino acids, and/or lipopeptides. For example, the present invention demonstrates that Bacillus subtilis can be grown in cell culture medium containing cellulosic material as a sole carbon source, and that production of lipopeptides by Bacillus subtilis in such medium is comparable or superior to production in medium containing glucose as a carbon source. According to the present invention, cellulosic material can be converted to high value products such as surfactants (e.g., acyl amino acid and lipopeptide surfactants) in cell culture. It has been shown that it is not necessary to provide exogenous carbohydrases in medium in which a cellulosic raw material such as soybean hulls is the carbon source (although, in some embodiments, it may be desirable to supply exogenous carbohydrases in medium).

In certain embodiments, microorganisms are grown in inventive cell culture medium that contain cellulosic material as a carbon source, which inventive cell culture medium further substantially lack an additional carbon source (e.g., the medium lack added glucose and glycerol). In certain embodiments, microorganisms are grown in inventive cell culture medium that contain cellulosic material as the sole carbon source. In certain embodiments, microorganisms grown in inventive cell culture medium that contain cellulosic material a carbon source produce one or more compounds of interest. For example, such microorganisms may produce polypeptides, peptides, acyl amino acids, and/or lipopeptides, which can be isolated and optionally purified from the cell culture. In certain embodiments, a cell culture medium includes cellulosic material as a carbon source. Cellulosic material is available from multiple sources. In some embodiments, cellulosic material is from industrial or agricultural waste, e.g., sawdust, paper mill sludge, paper pulp, wastepaper, fruit processing waste (e.g., citrus peel waste), and/or municipal solid waste. In some embodiments, cellulosic material is from plant material, e.g., leaves, stems and/or stalks. Examples of plant sources include soybeans, corn, wheat, sugarcane, trees, grasses (e.g., Miscanthus, Switchgrass, Bermuda grass, and Elephant grass).

Cellulose, a homologous polysaccharide comprised of long chains of glucose, is an abundant component of plant biomass, found primarily in plant cell walls. Cellulose fibers in plants are embedded in a matrix of other polymers, primarily hemicelluloses and lignin. Cellobiose is the smallest repeating unit of cellulose and can be converted into glucose. Hemicelluloses are heterologous polymers of five- and six-carbon sugars. Hemicelluloses can include pentoses (D-xylose, D-arabinose), hexoses (D-mannose, D-glucose, D-galactose) and sugar acids. In hardwoods, hemicellulose contains mainly xylans, while in softwood mainly glucomannans are present. Lignin is a complex aromatic polymer.

A cellulosic material for use in a culture medium as described herein may be provided in an unprocessed form (e.g., soybean hulls), in a decomposed form, and/or in a form enriched for a particular cellulosic component, such as xylose, cellobiose, or xylan. In some embodiments, cellulosic material is treated to release carbohydrates. Exemplary treatments include chemical (e.g., dilute acid, aqueous alkali treatment), mechanical, heat, and/or enzyme treatments. Dilute acid pretreatment is described in Grethlein, Bio/Technology 2:155-160, 1985; Schell et al., Appl. Biochem. Biotechnol. 77-79:67-81, 1999; and Torget, et al., Ind. Eng. Chem. Res. 39:2817-2825, 2000. Stem explosion treatment is described, e.g., in Brownell and Saddler, Biotechnol. Bioeng. 29:228-235, 1987; Heitz et al., Biores. Technol. 35:23-32, 1991; and Puls et al., Appl. Microbiol. Biotechnol. 22:416-423; 1985. Hydrothermal treatment is described, e.g., in Bobleter, Prog. Polym. Sci. 19:797-841, 1994; Laser et al., Biores. Technol. 81:33-44, 2002; and Mok and Antal. Ind. Eng. Chem. Res. 31:1157-1161, 1992. Organic solvent extraction is described, e.g., in Chum et al., Biotechnol. Bioeng. 31:643-649, 1988 and Holtzapple and Humphrey, Biotechnol. Bioeng. 26:670-676, 1984. Ammonia fiber explosion is described in Dale and Moriera, Biotechnol. Bioeng. Symp. Ser. 12:31-43, 1982. Sodium hydroxide treatment is described, e.g., in Weil et al., Enzyme Microb. Technol. 16:1002-1004, 1994. Lime treatment is described, e.g., in Chang et al., Appl. Biochem. Biotechnol. 63-65:3-19, 1997; and Kaar and Holtzapple, Biomass Bioenerg. 18:189-199, 2000. See also Wyman, Bioresour. Tech. 96(18):1959-66, 2005.

In some embodiments, cellulosic material is treated to release carbohydrates prior to use in a culture medium. In some embodiments, cellulosic material is treated in a culture medium (e.g., cellulosic material is provided in a culture medium with one or more enzymes that break down cellulosic material, e.g., cellulase, cellobiase, hemicellulase, and/or pectinase). In some embodiments, a cellulosic material is used which has not been treated to release carbohydrates (e.g., a cellulosic material is not treated with a carbohydrase).

In some embodiments, a culture medium includes a cellulosic material at 0.5%, 1%, 2%, 3%, 4%, 5%, 6%, 7%, 8%, 9%, 10%, 11%, 12%, 13%, 14%, or 15% w/v. In some embodiments, a culture medium includes soybean hulls at 1-10% w/v (e.g., 2%, 3%, 4%, 5%, 6%, 7%, 8%, 9%, or 10% w/v). In some embodiments, a culture medium includes cellobiose at 1-10% w/v (e.g., 2%, 3%, 4%, 5%, 6%, 7%, 8%, 9%, or 10% w/v). In some embodiments, a culture medium includes xylose at 1-10% w/v (e.g., 2%, 3%, 4%, 5%, 6%, 7%, 8%, 9%, or 10% w/v). In some embodiments, a culture medium includes xylan at 1-10% w/v (e.g., 2%, 3%, 4%, 5%, 6%, 7%, 8%, 9%, or 10% w/v).

In certain embodiments, a medium including cellulosic material as described herein is a medium for growing microorganisms (e.g., Bacillus) in which a carbon source such as glucose is substituted with cellulosic material. In certain embodiments, a medium including cellulosic material is a modified form of a medium described by Spizizen, Proc. Nat. Acad. Sci. USA 44(10):1072-0178, 1958. In certain embodiments, a medium including cellulosic material includes the following: (NH₄)₂SO₄, K₂HPO₄, KH₂PO₄, Na₃-citrate dehydrate, magnesium sulfate heptahydrate, CaCl₂ dihydrate, FeSO₄ heptahydrate, disodium EDTA dihydrate, and cellulosic material (e.g., soybean hulls, cellobiose, xylose, or xylan, at 2%, 3%, 4%, 5%, 6%, 7%, 8%, 9%, or 10% w/v. In certain embodiments, a medium including cellulosic material includes the following: (NH₄)₂SO₄ at 2 g/L, K₂HPO₄ at 14 g/L, KH₂PO₄ at 6 g/L, Na₃-citrate dihydrate at 1 g/L, magnesium sulfate heptahydrate at 0.2 g/L, CaCl₂ dihydrate at 14.7 mg/L, FeSO₄ heptahydrate at 1.1 mg/L, disodium EDTA dihydrate at 1.5 mg/L, and cellulosic material at 2%, 3%, 4%, 5%, 6%, 7%, 8%, 9%, or 10% w/v). Other media formulae suitable for growing microorganisms (e.g., Bacillus) such as Luria Bertani (LB) media are known and may be modified to include cellulosic material as a carbon source in accordance with the present invention.

### Production of Polypeptides, Non-ribosomal Peptides, Acyl Amino Acids, and Lipopeptides in Microorganisms Using Cellulosic Material as a Carbon Source

In certain embodiments, a microorganism grown in compositions of the present invention and/or according to methods of the present invention produces one or more products of interest. For example, a microorganism may produce a polypeptide, non-ribosomal peptide, acyl amino acid, and/or a lipopeptide. As one non-limiting example, a microorganism may produce the lipopeptide surfactin. Surfactin is cyclic lipopeptide that is naturally produced by certain bacteria, including the Gram-positive endospore-forming bacteria Bacillus subtilis. Surfactin is an amphiphilic molecule (having both hydrophobic and hydrophilic properties) and is thus soluble in both organic solvents and water. Surfactin exhibits exceptional surfactant properties, making it a commercially valuable molecule. Surfactin consists of a seven amino acid peptide loop, and a hydrophobic fatty acid chain (beta-hydroxy myristic acid) thirteen to fifteen carbons long. The fatty acid chain allows surfactin to penetrate cellular membranes. The peptide loop is composed of the amino acids glutamic acid, leucine, D-leucine, valine, aspartic acid, D-leucine and leucine. Glutamic acid and aspartic acid residues at positions 1 and 5 respectively, constitute a minor polar domain. On the opposite side, valine residue at position 4 extends down facing the fatty acid chain, making up a major hydrophobic domain.

Surfactin is synthesized by the linear nonribosomal peptide synthetase, surfactin synthetase, which includes three synthetase subunits SrfA-A, SrfA-B, and SrfA-C. Each of the enzymes SrfA-A and SrfA-B consist of three amino acid activating modules, while the monomodular subunit SrfA-C adds the last amino acid residue to the heptapeptide. Additionally the SrfA-C subunit includes the thioesterase domain (“TE domain”), which catalyzes the release of the product via a nucleophilic attack of the beta-hydroxy of the fatty acid on the carbonyl of the C-terminal Leu of the peptide, cyclizing the molecule via formation of an ester.

Due to its surfactant properties, surfactin also functions as an antibiotic. For example, surfactin is known to be effective as an anti-bacterial, anti-viral, anti-fungal, anti-mycoplasma and hemolytic compound. As an anti-bacterial compound, surfactin it is capable of penetrating the cell membranes of all types of bacteria, including both Gram-negative and Gram-positive bacteria, which differ in the composition of their membrane. Gram-positive bacteria have a thick peptidoglycan layer on the outside of their phospholipid bilayer. In contrast, Gram-negative bacteria have a thinner peptidoglycan layer on the outside of their phospholipid bilayer, and further contain an additional outer lipopolysaccharide membrane. Surfactin's surfactant activity permits it to create a permeable environment for the lipid bilayer and causes disruption that solubilizes the membrane of both types of bacteria. In order for surfactin to carry out minimal antibacterial effects, the minimum inhibitory concentration (MIC) is typically in the range of 12-50 μg/ml.

In addition to its antibacterial properties, surfactin also exhibits antiviral properties, and is known to disrupt enveloped viruses such as HIV and HSV. Surfactin not only disrupts the lipid envelope of viruses, but also their capsids through ion channel formations. Surfactin isoforms containing fatty acid chains with 14 or 15 carbon atoms exhibited improved viral inactivation, thought to be due to improved disruption of the viral envelope.

Certain acyl amino acids such as sodium cocoyl glutamate also have surfactant properties. Useful acyl amino acids such as acylated glutamate, and other acylated amino acids, can be produced using media and methods described herein.

Those of ordinary skill in the art will be aware of other products (e.g., polypeptides, non-ribosomal peptides, acyl amino acids, and/or a lipopeptides) that are produced by any of a variety of microorganisms and will be able to select an appropriate microorganism to produce a product (e.g., a polypeptide, non-ribosomal peptide, acyl amino acid, and/or a lipopeptide) of interest by growing such a microorganism in compositions of the present invention and/or in accordance with methods of the present invention. In certain embodiments, a microorganism is engineered to produce a product of interest. For example, in some embodiments, a microorganism is engineered to express a polypeptide(s) that participates in the synthesis of the product of interest. In some embodiments, the polypeptide is an engineered polypeptide. In some embodiments, a microorganism that produces an acyl amino acid includes an engineered polypeptide comprising a fatty acid linkage domain, a peptide synthetase domain, and a thioesterase domain. In some embodiments, a microorganism that produces an acyl amino acid includes an engineered polypeptide comprising a fatty acid linkage domain, a peptide synthetase domain, and a reductase domain. In various embodiments, one or more of the fatty acid linkage domain, the peptide synthetase domain, and the thioesterase domain are surfactin synthetase domains. Methods of producing lipopeptides and acyl amino acids using engineered polypeptides, and methods of producing microorganisms that include the polypeptides are described in WO 2008/131002 and WO 2008/131014, the entire contents of which are hereby incorporated by reference.

In certain embodiments, a microorganism used to produce a polypeptide, non-ribosomal peptide, acyl amino acid, and/or a lipopeptide of interest when grown in compositions of the present invention and/or in accordance with methods of the present invention is a bacterium. Non-limiting examples of bacteria that can be grown in accordance with the present invention include bacteria of the genera Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix and Actinosynnema. In certain embodiments, a microorganism used to produce a polypeptide, non-ribosomal peptide and/or a lipopeptide in accordance with the present invention is a bacterium of the genus Bacillus. In certain embodiments, a microorganism used to produce a polypeptide, non-ribosomal peptide, acyl amino acid, and/or a lipopeptide in accordance with the present invention is a bacterium of the species Bacillus subtilis. Those of ordinary skill in the art will be aware of other bacteria that can produce polypeptides, non-ribosomal peptides, acyl amino acids, and/or lipopeptides when grown in compositions of the present invention and/or in accordance with methods of the present invention.

In certain embodiments, a microorganism used to produce a product of interest when grown in compositions of the present invention and/or in accordance with methods of the present invention is a fungus. Non-limiting examples of fungi that can be grown in accordance with the present invention include yeast of the genera Saccharomyces, Pichia, Aspergillus, Trichoderma, Kluyveromyces, Candida, Hansenula, Schizpsaccaromyces, Yarrowia, and Chrysoporium. Those of ordinary skill in the art will be aware of other fungi that can produce products when grown in compositions of the present invention and/or in accordance with methods of the present invention. In certain embodiments, a microorganism used in accordance with the present invention is a yeast of the genus Saccharomyces. In certain embodiments, a microorganism used in accordance with the present invention is a yeast of the species Saccharomyces cerevisiae.

Saccharomyces cerevisiae is among the first cellular organisms utilized by humans and continues to serve as a model eukaryotic organism for biological research. The extensive level of biochemical characterization of Saccharomyces cerevisiae metabolism achieved to date is a result of a thorough understanding of growth and fermentation conditions as well as the ease with which this yeast organism can be genetically manipulated. These factors combine to make this yeast organism an ideal platform for bioengineering efforts.

Growth of Saccharomyces cerevisiae requires the presence of a carbon source to support metabolic functions. Dextrose (glucose) is the preferred carbon source under aerobic conditions as an overwhelming body of evidence supports the production of metabolites to high concentrations with its use (Barnett, J. A., Payne, R. W., and Yarrow, D., Yeasts: characteristics and identification, 1st Ed., Cambridge University Press, Cambridge, 1983). However, S. cerevisiae is capable of using a variety of fermentable and non-fermentable sugars as carbon sources, increasing the versatility of this organism as an industrial platform for chemical production (see for example, Grannot and Snyder, Carbon source induces growth of stationary phase yeast cells, independent of carbon source metabolism, Yeast, May; 9(5):465-79, 1993).

Methods and compositions of the present invention expand the utility of Saccharomyces cerevisiae and other microorganisms as industrial platforms for chemical production.

In certain embodiments, Saccharomyces cerevisiae is grown in a cell culture medium comprising cellulosic material as a carbon source. In certain embodiments, Saccharomyces cerevisiae is grown in a cell culture medium that comprises cellulosic material as an energy source, which cell culture medium further substantially lacks glucose or refined glycerol. In certain embodiments, Saccharomyces cerevisiae is grown in a cell culture medium that comprises cellulosic material as the sole energy source.

In certain embodiments, a composition of the present invention used to grow a microorganism that produces one or more polypeptides, non-ribosomal peptides, acyl amino acids, and/or a lipopeptides of interest comprises a complex cell culture medium. As recognized in the art, complex media typically contain at least one component whose identity or quantity is either unknown or uncontrolled. Non-limiting examples of components that may be added to complex media include yeast extract, bacto-peptone, and/or other hydrolysates. In certain embodiments, a microorganism grown in a complex medium of the present invention comprising cellulosic material (e.g., soybean hulls, cellobiose, xylose, xylan) as a carbon source produces a polypeptide, non-ribosomal peptide, acyl amino acid, and/or a lipopeptide of interest in an amount that is nearly the amount of the product that would be produced if the microorganism were grown under otherwise identical conditions in a traditional complex medium. For example, a polypeptide, non-ribosomal peptide, acyl amino acid, and/or a lipopeptide produced by a microorganism in accordance with the present invention may be produced in an amount that is at least 20%, 25%, 30%, 35%, 40%, 45%, 50%, 55%, 60%, 65%, 70%, 75%, 80%, 85%, 90%, 95%, 96%, 97%, 98%, 99% or more the amount of polypeptide, non-ribosomal peptide, acyl amino acid, and/or a lipopeptide that would be produced if the microorganism were grown under otherwise identical conditions in a traditional complex medium. In certain embodiments, a microorganism grown in a complex medium of the present invention comprising cellulosic material as a carbon source produces a polypeptide, non-ribosomal peptide, acyl amino acid, and/or a lipopeptide of interest in an amount that is equivalent to the amount that would be produced if the microorganism were grown under otherwise identical conditions in a traditional complex medium. In certain embodiments, a microorganism grown in a complex medium of the present invention comprising cellulosic material as a carbon source produces a polypeptide, non-ribosomal peptide, acyl amino acid, and/or a lipopeptide of interest in an amount that is greater than the amount of polypeptide, non-ribosomal peptide, acyl amino acid, and/or a lipopeptide that would be produced if the microorganism were grown under otherwise identical conditions in a complex defined medium.

In certain embodiments, a composition of the present invention used to grow a microorganism that produces one or more polypeptides, non-ribosomal peptides, acyl amino acids, and/or a lipopeptides of interest comprises a defined cell culture medium. A variety of chemically defined growth media for use in cell culture are known to those of ordinary skill in the art. Since each component of a defined medium is typically well characterized and present in known amounts, defined media do not contain complex additives such as serum or hydrolysates. Such defined media can be modified according to the teachings of the present disclosure to generate a cell culture medium that comprises cellulosic material as a carbon source. In certain embodiments, a defined medium of the present invention comprises cellulosic material as a carbon source, and further substantially lacks a second carbon source (e.g., the medium lacks glucose or glycerol). In certain embodiments, a defined medium of the present invention comprises cellulosic material as the sole carbon source.

In certain embodiments, a defined cell culture medium of the present invention comprises a limiting amount of one or more components. As one non-limiting embodiment, a cell culture medium of the present invention may comprise a limiting amount of nitrogen.

In certain embodiments, a microorganism grown in a defined or complex medium of the present invention comprising cellulosic material as a carbon source produces a polypeptide, non-ribosomal peptide, acyl amino acid, and/or a lipopeptide to a level of 40 mg/L, 50 mg/L, 75 mg/L, 100 mg/L, 125 mg/L, 150 mg/L, 175 mg/L, 0.2 g/L, 0.3 g/L, 0.4 g/L, 0.5 g/L, 0.6 g/L, 0.7 g/L, 0.8 g/L, 0.9 g/L, 1.0 g/L, 2.0 g/L, 3.0 g/L, 4.0 g/L, 5.0 g/L, 6.0 g/L, 7.0 g/L, 8.0 g/L, 9.0 g/L, 10 g/L, 20 g/L, 30 g/L, 40 g/L, 50 g/L, 60 g/L, 70 g/L, 80 g/L, 90 g/L, 100 g/L, or more.

In certain embodiments, the amount of polypeptide, non-ribosomal peptide, acyl amino acid, and/or lipopeptide of interest produced is increased by subjecting a cell culture containing a microorganism that produces the polypeptide, non-ribosomal peptide, acyl amino acid, and/or lipopeptide to one or more methods of the present invention. In certain embodiments, the production of a polypeptide, non-ribosomal peptide, acyl amino acid, and/or lipopeptide of interest is supplementing the cell culture with a nitrogen source such as without limitation, tryptone, total soy extract, yeast extract, casamino acids and/or distiller grains. In certain embodiments, a microorganism produces a polypeptide, non-ribosomal peptide, and/or lipopeptide of interest to an increased level relative to the level of polypeptide, non-ribosomal peptide, acyl amino acid, and/or lipopeptide that would be produced by a microorganism grown under otherwise identical conditions in an otherwise identical cell culture medium that lacks the provided nitrogen source. In certain embodiments, a nitrogen source added to the cell culture increases production of the polypeptide, non-ribosomal peptide, acyl amino acid, and/or lipopeptide of interest by a relatively greater amount than amount by which the total biomass of the cell culture is increased. In such embodiments, a polypeptide, non-ribosomal peptide, acyl amino acid, and/or lipopeptide of interest produced in a cell culture to which the nitrogen source is added represents an increased fraction of the total biomass of the cell culture compared the fraction that would result if the nitrogen source were not added to the cell culture.

In certain embodiments, the yield of a polypeptide, non-ribosomal peptide, acyl amino acid, and/or a lipopeptide of interest produced by a microorganism grown in inventive media containing cellulosic material is at least about 10%, 15%, 20%, 25%, 30%, 35%, 40%, 45%, 50%, 55%, 56%, 57%, 58%, 59%, 60%, 65%, 70%, 75%, 80%, 85%, 90% or more. Yield is defined as the amount of carbon source (e.g., cellobiose) that is converted to product (e.g., a polypeptide, non-ribosomal peptide, acyl amino acid, and/or a lipopeptide). Thus, if 50% of cellobiose is converted to a polypeptide, non-ribosomal peptide, acyl amino acid, and/or a lipopeptide, the yield is 50%.

In certain embodiments, a microorganism grown in a defined medium of the present invention comprising cellulosic material as a carbon source grows to a cell density that is comparable to the cell density that would be achieved if the microorganism were grown under otherwise identical conditions in a traditional defined medium. For example, a microorganism grown in a defined medium of the present invention comprising cellulosic material as a carbon source may grow to a cell density that is at least 50%, 55%, 60%, 65%, 70%, 75%, 80%, 85%, 90%, 95%, 96%, 97%, 98%, 99% or greater than the cell density that would be achieved if the microorganism were grown under otherwise identical conditions in a traditional defined medium. In certain embodiments, a microorganism grown in a defined medium of the present invention comprising cellulosic material as a carbon source grows to a cell density that is greater than the cell density that would be achieved if the microorganism were grown under otherwise identical conditions in a traditional defined medium. For example, a microorganism grown in a defined medium of the present invention comprising cellulosic material as a carbon source may grow to a cell density that is at least 100%, 110%, 120%, 130%, 140%, 150% or greater than the cell density that would be achieved if the microorganism were grown under otherwise identical conditions in a traditional defined medium.

### EXAMPLES

### Example 1

### Production of a Lipopeptide in Cellulosic Media

A genetically engineered mutant of a strain derived from the Bacillus Genetic Stock Center, named OKB105 (sfp Phe⁻), was used in the following experiments. This strain, which is a phenylalanine auxotroph, is capable of producing the seven amino acid lipopeptide surfactin (Nakano et al., J Bacteriol. 170(12):5662-8, 1988). The ability of this strain to synthesize phenylalanine was restored by transforming it with a linear piece of DNA that was PCR-amplified using as a template total genomic DNA of Bacillus subtilis 168. The PCR reaction was carried out using the following primers:

(SEQ ID NO: 1)

23848: 5′-TACATTGTTCTTGAATTAAAAGTGCTTGCAGATG-3′

(SEQ ID NO: 2)

23849: 5′-TCTGGCCATTCAATCATTGTTAAACG-3′

The resulting PCR product was cleaned using a PCR Purification kit (Qiagen) and used directly to transform OKB105 competent cells. The resulting transformants were selected in (SMM). A colony that was able to grow in that media was assigned the name 028836. This strain was utilized in the experiments described in this example.

Experiments were carried out utilizing a modified Spizizen's minimal media (MM15). Spizizen's (unmodified) minimal media (SMM) consists of ammonium sulfate 0.2%, dipotassium phosphate 1.4%, monopotassium phosphate 0.6%, sodium citrate dihydrate 0.1%, magnesium sulfate heptahydrate 0.02%, and glucose 0.5% (Spizizen, Proc. Nat. Acad. Sci. USA, 44(10):1072-8, 1958).

The protocol used for producing surfactin includes initially growing a “pre-seed” and inoculating the pre-seed into a seed culture, which is then inoculated into a “production” media. Both pre-seed and seed are grown for 24 hrs at 30° C. Production media is grown for 120 hrs at 30° C. The pre-seed and seed are used to inoculate seed, and production media at 2% vol/vol, respectively. The media composition of the pre-seed is M9YE +0.5% glycerol (M9YE: Na₂HPO₄ 6 g, KH₂PO₄ 3 g, NaCl 0.5 g, NH₄Cl 1 g, yeast extract 3 g, water to 990 ml). The media composition of the “seed” is (NH₄)₂SO₄ 2 g, K₂HPO₄ 14 g, KH₂PO₄ 6 g, Na₃-citrate dihydrate 1 g, magnesium sulfate heptahydrate 0.2 g, glucose 40 g, CaCl₂ dihydrate 14.7 mg, FeSO₄ heptahydrate 1.1 mg, disodium EDTA dihydrate 1.5 mg per liter of water. The “production” culture is obtained by inoculating 2% of “seed” into the “production” media, which is identical to the “seed” media plus 10 μM of MnSO₄. Using this protocol, surfactin was obtained at a concentration of 1.26 g/L after three days in the production media.

To investigate the performance of cellulosic material as a carbon source, the above protocol was repeated using the same pre-seed and seed, but replaced glucose in the production media with xylose at weight-to-volume ratios of 4%, 8%, 16%, and 42%, cellobiose at weight-to-volume ratios of 2%, 4%, 10%, and xylan at weight-to-volume ratios of 1%, 4%, and 10%. The evaluation of the effect of carbon sources on surfactin production was compared to glucose with a weight-to-volume ratio of 4%, the standard “production” culture carbon source percentage.

D-(+)-Xylose (catalog number X3877), D-(+)-cellobiose (catalog number 22150), and xylan from Birchwood (catalog number X0502) were purchased from Sigma-Aldrich. The stock solutions of xylose (50 g/100 mL), cellobiose (12 g/100 mL), and glucose (50 g/100 mL) were filter sterilized prior to addition to the “production” culture media, whereas the xylan was added to water and then autoclaved prior to the addition of the remaining components of the “production” culture media. One-milliliter of the “seed” culture was added to each 50 mL flask containing the modified MM15 media, a 2% innoculum. The flasks were shaken at 225-rpm at 30° C. for 48 hours prior to analysis for surfactin production using liquid-chromatography/mass spectrometry (LC/MS).

### Detection of Surfactin

Prior to injection into the LC/MS, 1 mL of each culture was centrifuged at 10,000×g for 5 minutes. The culture supernatant was diluted 1-to-20 in sterile deionized water and then filtered through a Millipore Ultrafree-MC 0.45 μm column during centrifugation at 5,000×g for 5 minutes. The LC system was comprised of a Thermo-Scientific Accela-autosampler, an Accela-pump, and an Accela-PDA detector. The Thermo Scientific C18-HPLC column Hypersil Gold was used for the resolution of surfactin from other media components. The mobile phase for the reverse phase resolution surfactin was 100% water (supplemented with 1% acetic acid) for 3 minutes, 100% water to 100% acetonitrile (supplemented with 1% acetic acid) in 7 minutes, 100% acetonitrile for 2 minutes, 100% isopropanol for 3 minutes, the LC system was re-equilibrated to 100% water for 4 minutes prior to the next LC/MS injection. After LC resolution was achieved, the MS detection of surfactin was performed using a Thermo Scientific LXQ in electrospray-negative mode. The MS method was programmed to capture from the first mass of m/z=100 to the last mass of m/z=1,200. Surfactin production was analyzed through the detection of the compound masses with the m/z ratios=992.7, 1006.7, 1020.7, 1034.7, and 1048.7.

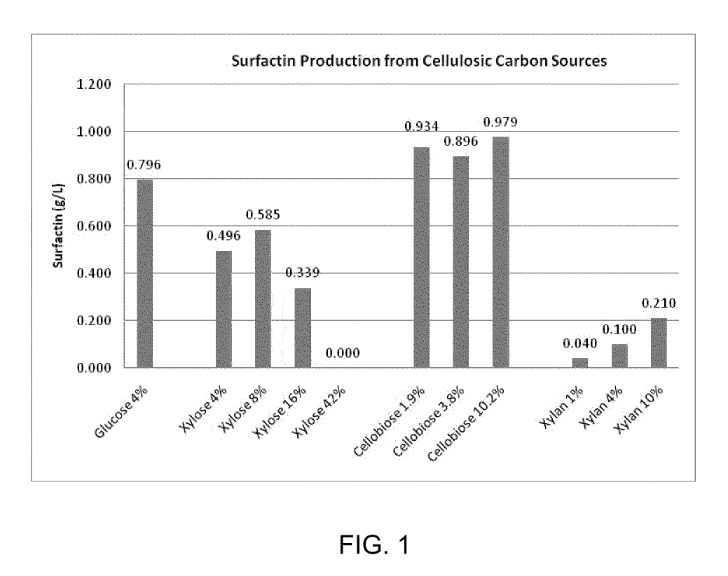

Surfactin production from cellulosic carbon sources as compared to glucose are shown in FIG. 1. As can be seen in FIG. 1, the greatest amount of surfactin production occurred during growth on 10% cellobiose (0.979 g/L), as compared to 4% glucose (0.796 g/L), 8% xylose (0.585 g/L), or 10% xylan (0.210 g/L).

### Example 2

### Production of an Acyl Amino Acid in Cellulosic Media

A wide variety of carbohydrate sources are available that can be used for commercial production of chemicals. Often, chemicals and biofuels are produced using carbohydrate that could enter the food supply (e.g. corn starch). Carbohydrate derived from cellulosic material is a preferred raw material for bio-production of chemicals. Cellulosic carbohydrate is not processed to generate food. Furthermore, it is the most abundant form of carbohydrate on earth. In the following experiments, it was determined whether cellulosic carbohydrate could be used in fermentation for production of FA-Glu.

### Materials and Methods

Strains and Media. OKB105 (pheA1 sfp) (Bacillus Genetic Stock Center, BGSC). Sure 2 (Stratagene) cells were used for all E. coli transformations. Plasmids pUC19 (New England Biolabs, NEB), and pDG364 (BGSC), which allows DNA integration at the AmyE locus, were used for vector constructions. Bacillus cells were grown in Luria-Bertani (LB), Spizizen minimal media (SMM) using 0.5% or 4% glucose supplemented with 100 μg/ml phenylalanine, 0.1 mM CaCl₂, 4 μM Fe(SO₄), 4 μM Na₂-EDTA, 10 μM MnSO₄ t 30° C. When needed, media was supplemented with kanamycin (30 μg/ml), spectinomycin (100 μg/ml), thymine (25 μg/ml). E. coli cells were grown in Circle Grow (QBiogene). Unless noted, all chemicals were obtained from Sigma.

Bacillus transformations. Competent cells were obtained. Selections for seamless chromosomal integrations were carried out following published protocols.

Preservation of cell competence. The ability of cells to bind and take up DNA was maintained by designing a construct in which ComS is under the regulation of the surfactin promoter. This construct was PCR-amplified and inserted in between the EcorI and HindIII sites of pDG364 and introduced into the chromosome of OKB105 at the amyE locus by double crossover recombination. The resulting strain was named OKB105-ComS (pheA1 sfp Psrf-ComS Cm^{R}).

Engineering of acyl-glutamate (FA-Glu). The protocol used for engineering of seamless chromosomal mutations required the deletion of the uracil phosphoribosyl transferase gene (upp) in OKB105-ComS. The resulting strain was named OKB105-ComS-Δupp (pheA1 sfp Psrf-ComS Cm^{R}Δupp). The following procedure for making a modification at a particular locus established the “marking” with upp and a kanamycin resistance gene (kan) the desired site of recombination. Accordingly, genomic DNA of OKB105-ComS was used as a template to amplify upp and its promoter using primers: UPP-5′-KpnI: 5′-GCTAGCGGTACCGGGTTTTTTGACGATGTTCTTGAAACTCAATG-3′ (SEQ ID NO: ______) and UPP-3′-BamHI: 5′-AACGTTGGATCCCAGAATGTTCACATTTTCACCTATAATTGTATACAG-3′ (SEQ ID NO: ______). This PCR product and pUC19 were digested with KpnI and BamHI, ligated with T4 DNA ligase, and transformed into Sure 2 cells. The resulting plasmid was named pUC19-UPP. A DNA fragment conferring resistance to kanamycin and originating from the streptococcal plasmid pJH1 was amplified using primers: KAN-5′-BamHI: 5′-ACATCAGGATCCGATAAACCCAGCGAACCATTTGAGGTGATAGG-3′ (SEQ ID NO: ______) and KAN-3′-SalI: 5′-TAGTATGTCGACCCAATCAAAAAACAGATGGCCGCTATTAAAGCAGG-3′ (SEQ ID NO: ______). The PCR product and pUC19-UPP were digested with BamHI and SalI, ligated, and transformed into Sure 2 cells. The resulting plasmid was named pUC19-UPP-KAN. A two-step procedure was used to make a seamless fusion between the module that encodes glutamic acid and the 3′-end of module 7, which encoded L-leucine. For the first step, ˜1 kb sequences homologous to the 3′-end of the glutamic acid module, and the 3′-end of SrfA-C and the 5′-end of SrfA-D, were introduced at the 5′-end and 3′-end, respectively of the UPP-KAN insert in pUC19-UPP-KAN as follows. Due to the high similarity that exists among modules, it was necessary to do nested PCR reactions to amplify genomic DNA sequences. The C-terminus of the module that encodes Glu was obtained by PCR-amplification of the genomic DNA of strain OKB105 using primers 026663: 5′-ATGATTACAGCTATCATGGGAATTTTAA-3′ (SEQ ID NO: ______) and 026670: 5′-GCGGTGAAGAAACAGGATACGTA-3′ (SEQ ID NO: ______). The resulting PCR product was used as a template for primers 026682:5′-GCAGATTGTACTGAGAGTGCACCATAmUACGCTCGGAACCTTGCCTACA-3′ (SEQ ID NO: ______) and 026690:5′-CTGTGCGGTATTTCACACCGmCGTCAAAGATCCCCGCCTTCTC-3′ (SEQ ID NO: ______). This fragment was annealed to the PCR product obtained from the template pUC19-UPP-KAN and primers: 026688:5′-GCGGTGTGAAATACCGCACAmGATGCGTAAGGAGAAAATACC-3′ (SEQ ID NO: ______) and 026680:5′-ATATGGTGCACTCTCAGTACAATCTGmCTCTGATGCCGCATAGTTAA-3′ (SEQ ID NO: ______) using a ligation independent cloning technique. The product of this annealing reaction was named pUC19-GLU-UPP-KAN. The 3′-end of SrfA-C and 5′-end of SrfA-D of the surfactin locus were amplified with primers: 026664:5′-ACGACGAACGGGAAAGTCAAT-3′ (SEQ ID NO: ______) and 026671:5′-ATTGTTCAAGAGCCCGGTAATCT-3′ (SEQ ID NO: ______). The PCR product of this reaction was used as a template for primers: 026683:5′-ACATCCGCAACTGTCCATACTCTmGGATTTCTTTGCGCTCGGAGGGCA-3′ (SEQ ID NO: ______) and 026691:5′-AGCTATGACCATGATTACGCCAAmGTGATAACCGCCTGCGGAAAGA-3′ (SEQ ID NO: ______). This fragment was annealed to the PCR product obtained from pUC19-GLU-UPP-KAN opened with primers: 026689:5′-CTTGGCGTAATCATGGTCATAGCmUGTTTCCTGTGTGAAATTGTTAT-3′ (SEQ ID NO: ______) and 026681:5′-CAGAGTATGGACAGTTGCGGATGmUACTTCAGAAAAGATTAGATGTCTAA-3′ (SEQ ID NO: ______). The product of this annealing reaction was named pUC19-GLU-UPP-KAN-TE. This plasmid was used to transform OKB105-ComS-Δupp. The resulting strain was named OKB105-ComS-Δ mod(2-7) upp⁺ kan^{R} (pheA1 sfp Psrf-ComS Cm^{R} upp⁺ kan^{R}). A seamless fusion between the carboxy terminus of the module that specific glutamic acid (module 1) and the amino terminus of module 7 was obtained using pUC19-GLU-TE, which was obtained by removing the sequences encoding upp and kan in pUC19-GLU-UPP-KAN-TE by using primers: pUC19-GLU-TE-sense 5′-AAGGCGGGGATCTTTGAmCGATTTCTTTGCGCTCGGAGGG-3′ (SEQ ID NO: ______) and pUC19-GLU-TE-ANTI: 5′-GTCAAAGATCCCCGCCTmUCTCAACGTTCAGCACGTCCTGC-3′ (SEQ ID NO: ______). The resulting PCR product was annealed and transformed into Sure 2 cells. The resulting plasmid was named pUC19-GLU-TE. This plasmid was used to transform competent OKB105-ComS-Δ mod(2-7) upp⁺ Kan^{R}. Cells were selected on minimal media containing 5-fluorouracil. Cells that were kanamycin sensitive and 5-fluorouracil resistant were selected and per products resulting from amplification of their genomic DNA using primers 026663 and 026683 were sequenced. Cells with the desired fusion were named OKB105-ComS-Δ mod(2-7) Δupp FA-Glu (pheA1 sfp Psrf-ComS Cm^{R} Δupp). In the text, this strain is referred to as the FA-Glu strain.

Purification of FA-Glu. OKB105-ComS-Δ mod(2-7) Δupp FA-Glu was grown in a 14-liter New Brunswick (Bioflo 110) fermentor that had a line connected to 10-gallon carboy to collect foam. Cells were grown in 8 liters of SMM with 4% glucose and supplements for 5 days to produce FA-Glu. Filtered air was kept at 5 liter/min for the first two days and then increased to 15 liters/min to increase foam production. At the end of a run, foam was centrifuged at 8,000 g to remove cells that were carried over to the carboy. Foam was then processed by an ultrafiltration apparatus (GEHealthcare, Piscataway, N.J.) using a 500 kDa cutoff membrane. Filtered foam was incubated with shaking with ˜60 g of Diaion HP-20 (10 g of resin per 100 mg of FA-Glu) for 4 hours. The mixture was then loaded onto an empty column. Resin was washed with three bed volumes of water. Bound FA-Glu was eluted with 100% methanol. The eluted crude fraction of FA-Glu was concentrated to 20 ml of methanol and mixed with an equal volume of 0.9% NaCl, and NaOH was added to make the pH 9.5. The mixture was then loaded onto a reparatory funnel, mixed with three volumes of chloroform (1:1:3:methanol:NaCl:chloroform) and allowed for phases to separate. The lower phase was discarded and the upper phase was adjusted with HCl to pH 2.0 and extracted using the Bligh and Dyer method (Biochem. Cell Biol. 37(8): 911-917, 1959), using 0.9% NaCl pH 2.0 instead of water. Once phases separated, the lower phase was saved and the upper phase was re-extracted with additional chloroform so the ratio 4:3:8:methanol:0.9% NaCl:chloroform was preserved. Lower phases were pooled and dried using a rotary evaporator.

FA-Glu quantitation. FA-Glu present in the foam was quantified using a Thermo-Scientific Accela UHPLC system coupled to a Thermo Scientific LXQ ion trap mass spectrometer with an ESI probe. Chromatographic separation was carried out using a Thermo Scientific C18 Hypersil Gold column (50×2.1 mm, particle size 1.9 μm) at a flow rate of 200 μL min⁻¹. The sample injection volume was 25 μL, and the column temperate was maintained at 25° C. Mobile phase A was water modified with 1% (v/v) of acetic acid, and mobile phase B was acetonitrile modified with 1% (v/v) of acetic acid. The following gradient elution profile was applied: 0-1 min, 100% A; 1-2 min, 100% A to 100% B, hold for 3 minutes, and then re-equilibrated to 100% A for 1 minute prior to the next LC/MS injection. After LC resolution was achieved, the surfactants were detected in the negative ion mode using the following MS parameters: ion source voltage—5.0 kV, capillary voltage—30V, capillary temperature 275° C., tube lens offset—125V, sheath gas 20 (arbitrary units) and auxiliary gas 5 (arbitrary units). Mass spectra were acquired over the scan range m/z 100-1200, and data were processed using Xcalibur 2.0.7 software. The FA-Glu molecules were detected at the retention time from 3.5-5 min with m/z 344.21, 358.22, 372.24, 386.25, 400.27 and 414.28. FA-Glu molecules with an additional hydroxyl group were observed at retention time from 3.2-4 min at m/z 360.20, 374.22, 388.23, 402.25, 416.26 and 430.28. Quantitative analysis was carried using a series of FA-Glu standards to construct a calibration curve by plotting the area under the chromatographic peak as a function of the standard concentration. Within a certain range of concentrations, this curve corresponded to the equation of a straight line. The concentration of unknown samples was determined by matching the peak area of FA-Glu molecules with that on the calibration curve.

Determination of Solubility. To measure solubility, each surfactant sample was dissolved in aqueous solution, while maintaining a pH of 10. Surfactant was added until a precipitate was observed. The solution was centrifuges for 30 minutes and filtered through a 0.2 μfilter. Total Organic Carbon (TOC) was measured using a Shimadzu total organic carbon analyzer, and the concentration of surfactant in the saturated solution was calculated using the TOC values.

Determination of Critical Micelle Concentration. Samples of FA-Glu and myristoyl glutamate (Ajinomoto Amisoft MS-11) were each dissolved in triple distilled water and the pH was adjusted to 10. Surface tension was measured at 25±1° C. using the Wilhelmy plate technique with a sandblasted platinum plate as the sensor coupled to a Cahn microbalance. The entire assembly was kept in a draft-free plastic cage at a temperature of 25±1° C.

Fermentation of Soybean Hulls. An OKB105-ComS-Δ mod(2-7) Δupp FA-Glu seed culture was grown to saturation in 50 ml M9YE media (42 mM Na₂HPO₄, 22 mM KH₂PO₄, 8 mM NaCl, 19 mM NH₄Cl, 0.3% Yeast Extract, 0.5% Glucose) supplemented with spectinomycin in a 250 mL flask with shaking at 30° C. for 3 days. The soybean hull cultures were established in 50 ml SMM supplemented with soybean hull (SBH) with or without cellulosic enzymes for simultaneous saccharification and fermentation (SSF). Control cultures contained 50 mL SMM supplemented with 4% Glucose without SBH. All cultures were established in duplicate. The seed culture was used to seed these cultures at 2% total volume. SBH was used as the sole carbon source at 2% and 8% w/v. SBH was added to individual flasks and 30 ml of water was added to each before autoclaving. The following enzymes were added at the start of the fermentation. Cellulase (Celluclast 1.5 L, Sigma-Aldrich, lot 128K1301, 800 EGU/g), Cellobiase aka β-Glucosidase (Novozyme 188, Sigma-Aldrich, lot 078K0709, 258 CBU/g), Hemicellulase (Sigma-Aldrich, lot 059K1534, 1500 U/g), Pectinase (Pectinex, Sigma-Aldrich, lot 088K1651, 10454 U/mL) were used in combination. Cultures contained either all four enzymes, all enzymes except pectinase, all enzymes except pectinase and hemicellulase, cellulase alone, or no enzymes. Cellulase was used at a concentration of 5.1 U/g SBH. Cellobiase was used at a concentration of 15.5 U/g SBH. Hemicellulase was used at a concentration of 13.8 U/g SBH. Pectinase was used at a concentration of 500 U/g. Samples were removed daily from the culture, centrifuged at 13,000 RPM for 5 minutes, supernatant filtered through a 0.45 micron filter and diluted 1:10 for LCMS analysis.

Fermentation of Cellobiose and Xylose. OKB105-ComS-Δ mod(2-7) Δupp FA-Glu was grown to saturation 50 ml M9YE media supplemented with spectinomycin in a 250 ml flask with shaking at 30° C. for 3 days. Carbon sources: Cellobiose (Fluka), Glucose, Xylan (Sigma-Aldrich), and Xylose (Sigma-Aldrich) were prepared as 10% stock solutions in water and autoclaved. Cultures were established in 10 ml SMM supplemented with varying carbon sources. The M9YE culture was used to seed the cultures at 2% total volume. Carbon sources were tested at three concentrations, 0.5%, 2.0% and 8.0%. All cultures were done in quintuplicate. Cultures were incubated with shaking at 30° C. for 4 days. Samples were removed from culture, centrifuged at 13,000 RPM for 5 minutes, supernatant was filtered through a 0.45 micron filter and diluted 1:10 for LCMS analysis.

### Results

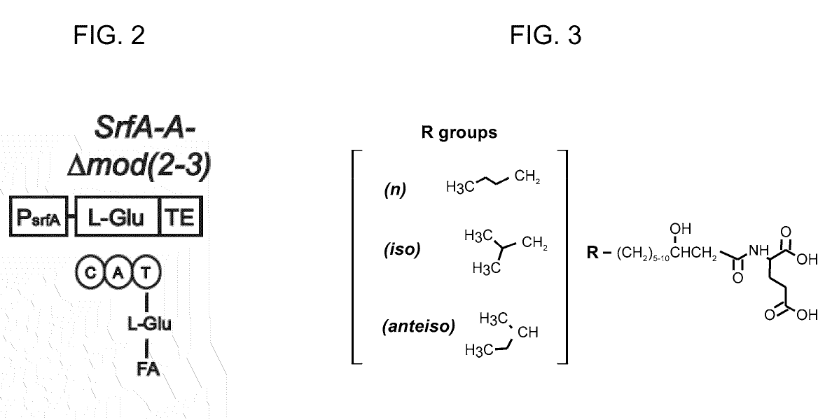

Microbial Strain Engineering. In order to produce a water soluble surfactant, the size of a surfactin synthetase was radically reduced with the goal of eliminating all of the enzyme-modules that specify the incorporation of hydrophobic amino acids into surfactin. This involved precise site-specific deletion of about 21 kilobases (kb) of the Bacillus genome in order to make the gene variant shown in FIG. 2. In this engineered Bacillus strain, the first module of the synthetase is fused to the thiolation domain of module 7 followed by the thioesterase domain. It was hypothesized that the engineered synthetase would produce an acyl amino acid composed of a β-hydroxy fatty acid (usually myristic) linked to glutamate, hereafter referred to as FA-Glu (Fatty Acid-Glutamate) (FIG. 3). It was anticipated that the molecule would not be cyclic given that steric constraints would likely limit the ability of the glutamate residue to link to the β position of the fatty acid via a lactone bond. It was assumed that the fatty acid of FA-Glu would be a mixture of straight-chain and branched species.

Fermentation and LCMS Analysis. Surfactin is produced by actively growing cells subsequent to the exponential phase of cell growth (Vater, Progr. Colloid & Polymer Sci. 72:12-18, 1986). It is secreted into the culture medium and causes the production of foam. Surfactin can be partially purified by methods such as foam fractionation (Cooper et al., Appl. Envoron. Microb. 42:408-412, 1981).

The engineered strain that produces FA-Glu is referred to as the FA-Glu strain in this example. The FA-Glu strain was grown in a shake flask under culture conditions similar to those used to support production of surfactin by the wildtype strain (Wei et al., Biotechnol. Lett. 24:479-482, 2002). The kinetics of FA-Glu production were essentially identical to the kinetics of surfactin production. The titer of FA-Glu is consistently about 1/10 that of the parent molecule (surfactin) on a weight basis. A volumetric productivity of 20 mg/L/day of FA-Glu is typical for the FA-Glu strain under conditions where the wildtype strain produces 400 mg/L/day of surfactin. This is not surprising given that engineered peptide synthetase and polyketide synthase enzymes often exhibit lower productivity than the natural enzymes (Stevens et al., Drug Dev. Res. 66:9-18, 2006).

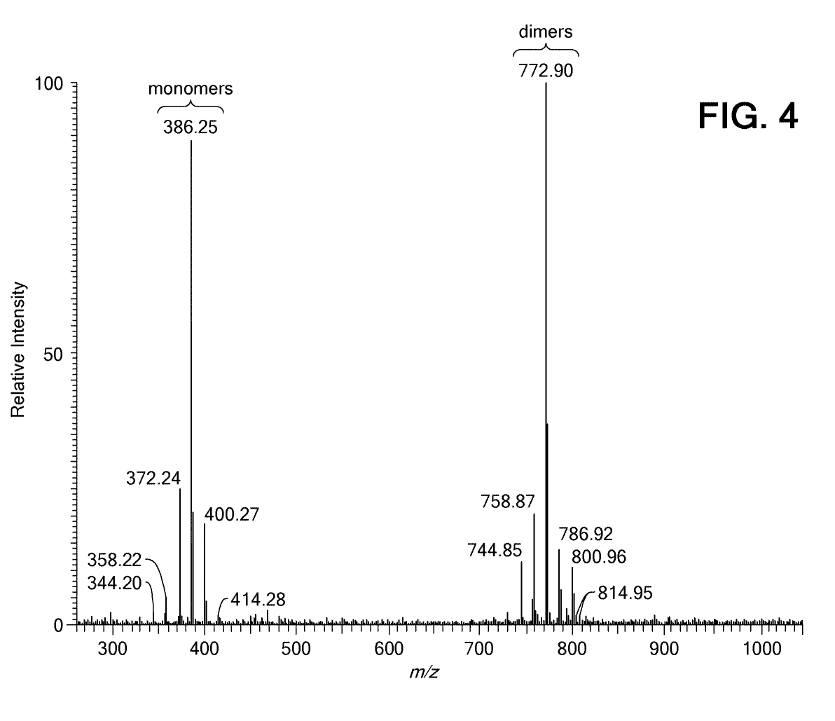

LCMS analysis showed that the surfactin-derivative (referred to as FA-Glu) could be detected in the culture media after about 24 hours of fermentation. Maximal production was seen after about three days. Production of FA-Glu produced foam, which enabled partial purification of FA-Glu by foam fractionation. The LCMS analysis identified both monomer and dimer forms of FA-Glu (FIG. 4).

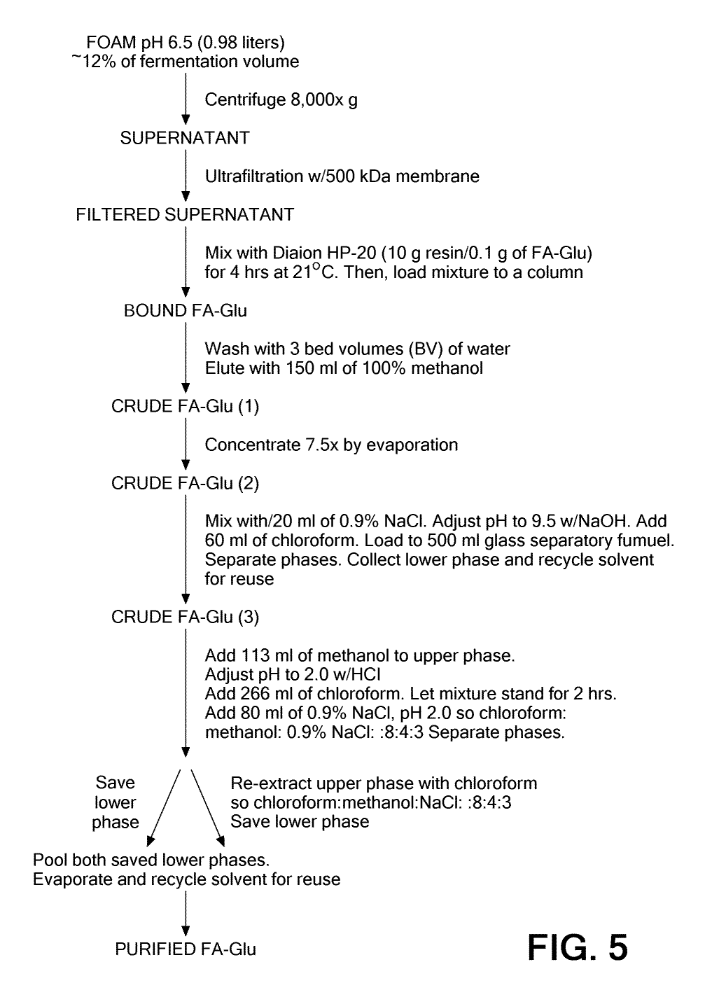

Purification of FA-Glu. The scheme used to purify FA-Glu is shown in FIG. 5. Fermentation is done in a fermentor and foam generated during the fermentation is allowed to escape the fermentor and accumulate in a plastic vessel. About 12% of the fermentor volume typically escapes as foam and we find that this fraction harbors nearly all of the FA-Glu. Centrifugation is used to remove cells from the condensed foam, followed by ultrafiltration and binding to Diaion HP-20 resin. The resin is then washed and FA-Glu is subsequently eluted using methanol. Purity of the FA-Glu sample is determined by comparing the yield estimated by quantitative LCMS analysis to the actual weight of a dried sample of purified FA-Glu. Using this method, it was observed that the FA-Glu obtained is typically about 30% pure.

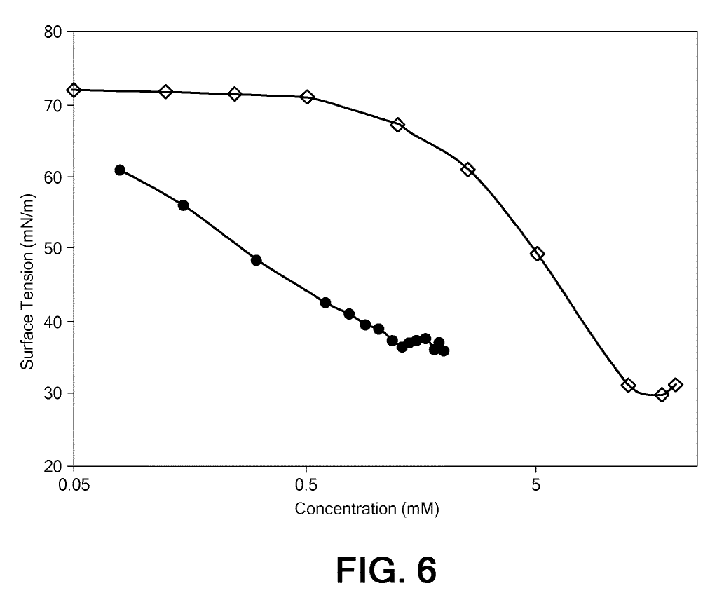

Measurement of Solubility and Critical Micelle Concentration (CMC). FA-Glu is very similar to a commercial surfactant that is widely used in consumer product formulations, myristoyl glutamate. Acyl amino acid surfactants, such as myristoyl glutamate, are popular with consumers because these surfactants interact favorably with skin and hair, are hypoallergenic, do not cause eye irritation, and are readily biodegradable (Nnanna et al., CRC Press Taylor & Francis Group, Oxfordshire, UK, 2001; Sakamoto, CRC Press Taylor & Francis Group, Oxfordshire, UK, 2001; Husmann et al., SOFW J. 130:22-28, 2004; Infante et al., Marcel Dekker, Inc., New York, USA, 2003). Given the similarity of FA-Glu to myristoyl glutamate, the water solubility and CMC of these surfactants were compared. It was observed that FA-Glu is more water soluble than myristoyl glutamate. FA-Glu is soluble to a concentration of 312 mM while myristoyl glutamate is soluble to a concentration of 89 mM. In addition, FA-Glu has higher surface activity, as reflected by its lower CMC (1.3 mM for FA-Glu versus 14.1 mM for myristoyl glutamate) (FIG. 6). A lower relative CMC indicates that less FA-Glu should be required in a formulation to achieve a particular desired reduction in surface tension. In addition, a lower CMC is correlated with an increased effectiveness in removing soils in cleaning formulation (Husmann et al., SOFW J. 130:22-28, 2004).

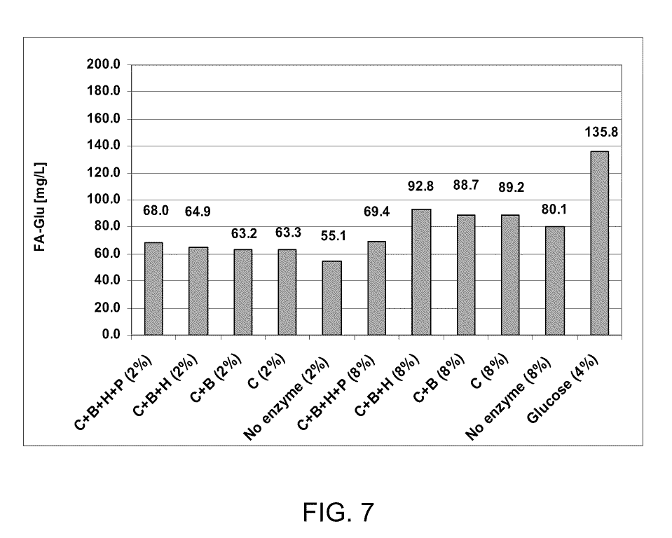

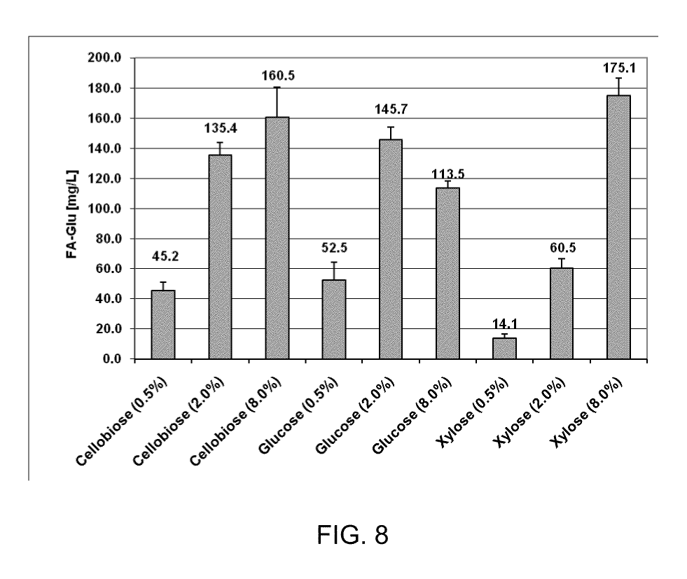

Production of FA-Glu using Cellulosic Carbohydrate. Soy hulls, an abundant agricultural waste material, were used as a carbon source in fermentation. Several different enzyme treatment strategies were compared, based on the assumption that the addition of exogenous carbohydrases would enhance FA-Glu production. The enzyme treatments were similar to those described by Mielenz and coworkers (Mielenz et al., Bioresource Technol. 100:3532-3539, 2009). In addition to soy hulls, samples of purified cellulose-derived carbohydrates were tested as the sole carbon source. FA-Glu was produced in all cases (FIGS. 7 and 8). Enzyme treatment of the soy hulls did not significantly increase FA-Glu production when compared to the “no enzyme controls”, indicating that Bacillus subtilis is able to utilize the cellulosic material even in the absence of enzyme treatment (FIG. 7). Bacillus subtilis strain 168 secretes xylanases and cellulases. These naturally occurring enzymes may enable utilization of the cellulosic material without the need for addition of exogenous enzymes. Interestingly, the titer of FA-Glu measured when either 8% cellobiose or 8% xylose was used as the sole carbon source was higher than the titer observed using 8% glucose (FIG. 8), indicating that cellulose-derived carbohydrate is a viable feedstock for FA-Glu production.

The ability to use this approach to generate a variety of surfactants provides an opportunity to broadly replace surfactants that are in wide use today with new molecules that can be generated via fermentation of biomass, such as cellulosic material derived from agricultural residue. Large-scale production of surfactants by fermentation of cellulosic material would reduce greenhouse gas emissions, while generating surfactants that are known to interact favorably with human skin, hair and eyes, and that are readily biodegradable. Significantly, the addition of exogenous enzymes, such as cellulase or xylanase, is not required for conversion of cellulosic material into FA-Glu by fermentation, which will reduce the cost of manufacturing of these surfactants.

The foregoing description is to be understood as being representative only and is not intended to be limiting. Alternative methods and materials for implementing the invention and also additional applications will be apparent to one of skill in the art, and are intended to be included within the accompanying claims.

1. A cell culture medium for growing Bacillus cells, the cell culture medium comprising a carbon source which comprises cellulosic material.

2. The cell culture medium of claim 1, wherein the cellulosic material comprises soybean hulls.

3. The cell culture medium of claim 1, wherein the cellulosic material comprises a composition enriched for cellobiose, xylose, xylan, or a combination thereof.

4. The cell culture medium of claim 3, wherein the cellulosic material comprises cellobiose.

5. The cell culture medium of claim 1, wherein the medium includes less than 0.1% glucose.

6. The cell culture medium of claim 2, wherein the medium lacks a carbon source other than the cellulosic material.

7. The cell culture medium of claim 2, wherein the medium comprises a cellulosic material at a weight to volume ratio of 1-10%.

8. The cell culture medium of claim 7, wherein the medium comprises the cellulosic material at a weight to volume ratio of 2-8%.

9. The cell culture medium of claim 1, wherein the medium is a liquid medium.

10. The cell culture medium of claim 1, wherein the medium is a solid medium.

11. The cell culture medium of claim 1, wherein the medium comprises (NH₄)₂SO₄, K₂HPO₄, KH₂PO₄, Na₃-citrate dihydrate, magnesium sulfate heptahydrate, CaCl₂ dihydrate, FeSO₄ heptahydrate, and disodium EDTA dihydrate.

12-15. (canceled)

16. The cell culture medium of claim 1, wherein the medium lacks exogenous carbohydrases.

17. The cell culture medium of claim 1, wherein the medium comprises an exogenous carbohydrase.

18. The cell culture medium of claim 18, wherein the exogenous carbohydrase comprises one or more of cellulase, cellobiase, hemicellulase, and pectinase.

19. A method for growing Bacillus cells in a cell culture, the method comprising growing the cells in a cell culture medium comprising a carbon source which comprises cellulosic material.

20. The method of claim 19, wherein the cellulosic material comprises soybean hulls.

21. The method of claim 19, wherein the cellulosic material comprises a composition enriched for cellobiose, xylose, xylan, or a combination thereof.

22. The method of claim 19, wherein the cellulosic material comprises cellobiose.

23. The method of claim 19, wherein the medium includes less than 0.1% glucose.

24. The method of claim 19, wherein the medium lacks a carbon source other than the cellulosic material.

25. The method of claim 19, wherein the Bacillus cells are Bacillus subtilis cells.

26. The method of claim 19, wherein the Bacillus cells produce a lipopeptide or an acyl amino acid.

27. The method of claim 26, wherein the Bacillus cells comprise a recombinant polypeptide which produces the lipopeptide or acyl amino acid.

28. The method of claim 27, wherein the recombinant polypeptide produces acyl glutamate.

29. The method of claim 26, wherein the cells produce a lipopeptide which comprises surfactin.

30. The method of claim 29, wherein the yield of surfactin produced from the cell culture is at least about 40 mg/L, 50 mg/L, 75 mg/L, 100 mg/L, 0.2 g/L, 0.3 g/L, 0.5 g/L, 0.7 g/L, 0.9 g/L or 1 g/L.

31. The method of claim 26, wherein the medium has less than 0.1% glucose, and wherein the cell culture produces a lipopeptide or acyl amino acid at a level at least comparable to a level of the lipopeptide or acyl amino acid produced in a culture in a medium having added glucose and which is otherwise identical to the medium comprising a carbon source which comprises the cellulosic material.

32. The method of claim 19, wherein the medium comprises a cellulosic material at a weight to volume ratio of 1-10%.

33-40. (canceled)

41. A method of producing a lipopeptide or an acyl amino acid, the method comprising:
providing a cell culture by growing Bacillus cells that produce a lipopeptide or an acyl amino acid in a cell culture medium, wherein the medium comprises a carbon source which comprises cellulosic material; thereby producing a lipopeptide or acyl amino acid.

42. The method of claim 41, further comprising isolating a portion of the cell culture which comprises the lipopeptide or acyl amino acid.

43. A method of producing a lipopeptide or an acyl amino acid, the method comprising:
providing a first cell culture by growing Bacillus cells that produce a lipopeptide or an acyl amino acid in a first cell culture medium, wherein the first medium comprises glycerol or glucose as a carbon source;
providing a second cell culture by inoculating a second cell culture medium with a portion of the first cell culture, wherein the second medium comprises cellulosic material as a carbon source;
thereby producing a lipopeptide or acyl amino acid.

44. The method of claim 43, wherein the first cell culture is grown for about 24 hours prior to inoculating the second culture.

45. A composition comprising Bacillus cells and cell culture medium, wherein the cell culture medium comprises a carbon source which comprises cellulosic material.

46. The composition of claim 45, wherein the cellulosic material comprises one or more of soybean hulls, cellobiose, xylose, or xylan.

47-48. (canceled)


SEQUENCE LISTING (20 sequences)

SEQ ID NO: 1 - DNA, 34 bp, Artificial Sequence
  Primer
tacattgttcttgaattaaaagtgcttgcagatg

SEQ ID NO: 2 - DNA, 26 bp, Artificial Sequence
  Primer
tctggccattcaatcattgttaaacg

SEQ ID NO: 3 - DNA, 44 bp, Artificial Sequence
  Primer
gctagcggtaccgggttttttgacgatgttcttgaaactcaatg

SEQ ID NO: 4 - DNA, 48 bp, Artificial Sequence
  Primer
aacgttggatcccagaatgttcacattttcacctataattgtatacag

SEQ ID NO: 5 - DNA, 44 bp, Artificial Sequence
  Primer
acatcaggatccgataaacccagcgaaccatttgaggtgatagg

SEQ ID NO: 6 - DNA, 47 bp, Artificial Sequence
  Primer
tagtatgtcgacccaatcaaaaaacagatggccgctattaaagcagg

SEQ ID NO: 7 - DNA, 28 bp, Artificial Sequence
  Primer
atgattacagctatcatgggaattttaa

SEQ ID NO: 8 - DNA, 23 bp, Artificial Sequence
  Primer
gcggtgaagaaacaggatacgta

SEQ ID NO: 9 - DNA, 48 bp, Artificial Sequence
  Primer
gcagattgtactgagagtgcaccatanacgctcggaaccttgcctaca

SEQ ID NO: 10 - DNA, 42 bp, Artificial Sequence
  Primer
ctgtgcggtatttcacaccgngtcaaagatccccgccttctc

SEQ ID NO: 11 - DNA, 41 bp, Artificial Sequence
  Primer
gcggtgtgaaataccgcacanatgcgtaaggagaaaatacc

SEQ ID NO: 12 - DNA, 46 bp, Artificial Sequence
  Primer
atatggtgcactctcagtacaatctgntctgatgccgcatagttaa

SEQ ID NO: 13 - DNA, 21 bp, Artificial Sequence
  Primer
acgacgaacgggaaagtcaat

SEQ ID NO: 14 - DNA, 23 bp, Artificial Sequence
  Primer
attgttcaagagcccggtaatct

SEQ ID NO: 15 - DNA, 47 bp, Artificial Sequence
  Primer
acatccgcaactgtccatactctngatttctttgcgctcggagggca

SEQ ID NO: 16 - DNA, 45 bp, Artificial Sequence
  Primer
agctatgaccatgattacgccaantgataaccgcctgcggaaaga

SEQ ID NO: 17 - DNA, 46 bp, Artificial Sequence
  Primer
cttggcgtaatcatggtcatagcngtttcctgtgtgaaattgttat

SEQ ID NO: 18 - DNA, 49 bp, Artificial Sequence
  Primer
cagagtatggacagttgcggatgnacttcagaaaagattagatgtctaa

SEQ ID NO: 19 - DNA, 39 bp, Artificial Sequence
  Primer
aaggcggggatctttgangatttctttgcgctcggaggg

SEQ ID NO: 20 - DNA, 40 bp, Artificial Sequence
  Primer
gtcaaagatccccgcctnctcaacgttcagcacgtcctgc